(19) United States
(12) Patent Application Publication
(10) Pub. No.: US 2025/0381217 A1
(43) Pub. Date: Dec. 18, 2025

(54) SELF-HEATING SKIN THERAPY SYSTEMS

(71) Applicant: ParaffinUSA, LLC, Phoenix, AZ (US)

(72) Inventor: Deanna Montrose, Phoenix, AZ (US)

(21) Appl. No.: 19/236,394

(22) Filed: Jun. 12, 2025

Related U.S. Application Data
(60) Provisional application No. 63/660,161, filed on Jun. 14, 2024.

(51) Int. Cl. A61K 31/74; A61F 7/00; A61K 8/92; A61K 9/00; A61Q 19/00

(52) U.S. Cl. CPC A61K 31/74; A61F 7/0085; A61K 8/92; A61K 9/0014; A61Q 19/007; A61F 2007/0036; A61K 2800/87

(57) ABSTRACT

A self-heating skin therapy system includes a body part shaped encaser having an interior space, an encaser liner disposed in the interior space of the encaser, and at least one warmer layer disposed on an outer surface of the encaser. The encaser liner has an internal space configured to receive the body part, and an inner surface of the encaser and an outer surface of the encaser liner are configured to define a first fillable space. An inner surface of the warmer layer and the outer surface of the warmer layer are configured to define a second fillable space. A therapeutic composition is contained within the first fillable space, and a warmer material is contained in the second fillable space. The warmer layer is permeable to air and the warmer material produces heat when exposed to air.

### CROSS REFERENCE TO RELATED APPLICATIONS

The present application claims priority to the Provisional Patent Application No. 63/660,161 filed on Jun. 14, 2024, disclosure of which is incorporated herein by reference in its entirety.

### BACKGROUND

Wax-based therapeutic compositions applied externally to the skin may be used to condition and soften skin, relieve joint pain, and improve a variety of skin conditions. Wax-based therapeutic compositions may be heated to melt the wax and to heat the skin or body part to which it is applied for pain relief. Current methods of melting bulk quantities of wax-based therapeutic compositions may take approximately two to three hours, impeding the delivery of the therapy to a person. Further, repeated heating-cooling cycles of wax-based therapeutic compositions for skin therapy may reduce the effectiveness of the composition over time. Additionally, overheating and uneven heating and cooling of the wax-based therapeutic compositions poses a significant safety risk for burning the person's skin.

### SUMMARY

The self-heating therapy system of the disclosed invention provides warmer material layer that is configured to produce heat when exposed to air. When the self-heating therapy system is removed from a package, the warmer material layer, being activated by exposure to air, provides heat to the therapeutic composition or skincare composition inside the self-heating therapy system. Therefore, additional process of heating the therapy system, for example by using a microwave oven, is not required.

These advantages and others are achieved, for example, by a self-heating skin therapy system for skin conditioning or treatment of a body part. The self-heating skin therapy system includes a body part shaped encaser having an interior space, an encaser liner disposed in the interior space of the encaser, at least one warmer layer disposed on an outer surface of the encaser, a therapeutic composition contained within the first fillable space, and a warmer material contained in the second fillable space. The encaser liner has an internal space configured to receive the body part, and an inner surface of the encaser and an outer surface of the encaser liner are configured to define a first fillable space. An inner surface of the warmer layer and the outer surface of the encaser are configured to define a second fillable space. The encaser liner is permeable to one or more ingredients of the therapeutic composition. The warmer layer is permeable to air and the warmer material produces heat when exposed to air.

The encaser may be impermeable to both the therapeutic composition and the warmer material. The at least one warmer layer may be attached on the outer surface of the encaser. The self-heating skin therapy system may further include a peripheral seal by which edges of the encaser and the encaser liner are joined together. The at least one warmer layer may comprise a plurality of patches attached on the outer surface of the encaser, and each patch defines a warmer fillable space filled with the warmer material. The patches may have shapes of a strip shape, rectangular shape, circular shape, or oval shape. The self-heating skin therapy system may further include a peripheral seal by which edges of the encaser, the encaser liner and the warmer layer are joined together. The warmer material may include a mixture of one or more of iron powder, water, salt, activated charcoal and wood fiber. The encaser liner may include non-woven polypropylene fabric. The warmer layer may include cotton cloth or non-woven polypropylene fabric. The body part shaped encaser has a glove shape, boot shape, or a mitt shape.

These advantages and others are achieved, for example, by a self-heating skin therapy system for skin conditioning or treatment of a body part. The self-heating skin therapy system includes a body part shaped encaser having an internal space configured to receive the body part, at least one warmer layer disposed on an outer surface of the encaser, a skincare composition contained within the internal space; and a warmer material contained in the fillable space. An inner surface of the warmer layer and the outer surface of the encaser are configured to define a fillable space. The warmer layer is permeable to air and the warmer material produces heat when exposed to air.

The encaser may be impermeable to both the skincare composition and the warmer material. The at least one warmer layer may be attached on the outer surface of the encaser. The at least one warmer layer may include a plurality of patches attached on the outer surface of the encaser, and each patch defines a warmer fillable space filled with the warmer material. The self-heating skin therapy system may further comprise a peripheral seal by which edges of the encaser and the warmer layer are joined together.

These advantages and others are achieved, for example, by a self-heating skin therapy system for skin conditioning or treatment of a body part. The self-heating skin therapy system includes a body part shaped encaser, an encaser liner disposed below the encaser, at least one warmer layer disposed above the encaser, a composition contained within the first fillable space, and a warmer material contained in the second fillable space. A lower surface of the encaser and an upper surface of the encaser liner are configured to define the first fillable space. A lower surface of the encaser liner is configured to contact a skin of a user. A lower surface of the warmer layer and an upper surface of the encaser are configured to define the second fillable space. The encaser liner is permeable to one or more ingredients of the composition, and the warmer layer is permeable to air and the warmer material produces heat when exposed to air.

The encaser may be impermeable to both the therapeutic composition and the warmer material. The at least one warmer layer may be attached on the outer surface of the encaser. The at least one warmer layer may include a plurality of patches attached on the outer surface of the encaser, and each patch defines a warmer fillable space filled with the warmer material. The self-heating skin therapy system may further include a peripheral seal by which edges of the encaser and the encaser liner are joined together. The body part shaped encaser has a shape suitable to cover face, elbow or knee of a user.

### BRIEF DESCRIPTION OF THE DRAWINGS

The preferred embodiments described herein and illustrated by the drawings hereinafter are included to illustrate and not to limit the invention, where like designations denote like elements.

FIG. 1A is an illustration of a first embodiment of a self-heating therapy system of the disclosed invention, which includes a glove shape encaser structure.

FIG. 1B is an illustration of a self-heating therapy system of the disclosed invention, which includes a boot shape encaser structure.

FIG. 1C is an illustration of a self-heating therapy system of the disclosed invention, which includes a mitt shape encaser structure.

FIG. 2 shows a cross-sectional view cut along A-A′ of the self-heating therapy systems shown in FIGS. 1A-1C.

FIG. 3 shows a layer structure of the section B of FIG. 2.

FIGS. 4A-4B show embodiments of the self-heating therapy systems including a glove shape encaser structure.

FIGS. 4C-4D show embodiments of the self-heating therapy systems including a boot shape encaser structure.

FIGS. 4E-4F show embodiments of the self-heating therapy system of the disclosed invention, which includes a mitt shape encaser structure.

FIG. 5 shows a cross-sectional view cut along C-C′ of the self-heating therapy systems shown in FIGS. 4A-4F.

FIG. 6A shows another embodiment of the self-heating therapy system including a glove shape encaser structure.

FIG. 6B shows a cross-sectional view cut along E-E′ of the self-heating therapy system shown in FIG. 6A.

FIG. 7A is an illustration of a second embodiment of self-heating therapy system of the disclosed invention, which includes a glove shape encaser structure.

FIG. 7B is an illustration of a self-heating therapy system of the disclosed invention, which includes a boot shape encaser structure.

FIG. 7C is an illustration of an embodiment of self-heating therapy system of the disclosed invention, which includes a mitt shape encaser structure.

FIG. 8 shows a cross-sectional view cut along G-G′ of the self-heating therapy systems shown in FIGS. 7A-7C.

FIG. 9 shows a layer structure of the section H of FIG. 8.

FIGS. 10A-10B show embodiments of the self-heating therapy systems including a glove shape encaser structure.

FIGS. 10C-10D show embodiments of the self-heating therapy systems including a boot shape encaser structure.

FIGS. 10E-10F show embodiments of the self-heating therapy systems including a mitt shape encaser structure.

FIG. 11 shows a cross-sectional view cut along K-K′ of the self-heating therapy systems shown in FIGS. 10A-10F.

FIG. 12A shows another embodiment of the self-heating therapy systems

FIG. 12B shows a cross-sectional view cut along M-M′ of the self-heating therapy system shown in FIG. 12A.

FIG. 13 is an illustration of a third embodiment of self-heating therapy system of the disclosed invention including a face mask shape encaser structure.

FIG. 14 is an illustration of an embodiment of self-heating therapy system of the disclosed invention including a knee or elbow shape encaser structure.

FIG. 15 shows a cross-sectional view cut along O-O′ of the self-heating therapy systems shown in FIGS. 13-14.

FIG. 16 shows layers of the section P of FIG. 15.

FIGS. 17A-17B are illustrations of another embodiment of the self-heating therapy system of the disclosed invention including a face mask shape encaser structure.

FIG. 18 is an illustration of another embodiment of the self-heating therapy system including a knee or elbow shape encaser structure.

FIG. 19 shows a cross-sectional view cut along Q-Q′ of the self-heating therapy systems shown in FIGS. 17-18.

FIG. 20 is an illustration of a fourth embodiment of self-heating therapy system of the disclosed invention including a face mask shape encaser structure and a warmer pack.

FIG. 21 is an illustration of an embodiment of self-heating therapy system of the disclosed invention including a knee or elbow shape encaser structure and a warmer pack.

FIGS. 22A-22B show cross-sectional views cut along S-S′ of the self-heating therapy systems shown in FIGS. 20-21.

FIG. 23 shows layers of the section T of FIG. 22B.

### DETAILED DESCRIPTION

The following detailed description is merely exemplary in nature and is not intended to limit the described embodiments or the application and uses of the described embodiments. All of the implementations described below are exemplary implementations provided to enable persons skilled in the art to make or use the embodiments of the disclosure and are not intended to limit the scope of the disclosure, which is defined by the claims. It is also to be understood that the drawings included herewith only provide diagrammatic representations of the presently preferred structures of the present invention and that structures falling within the scope of the present invention may include structures different than those shown in the drawings.

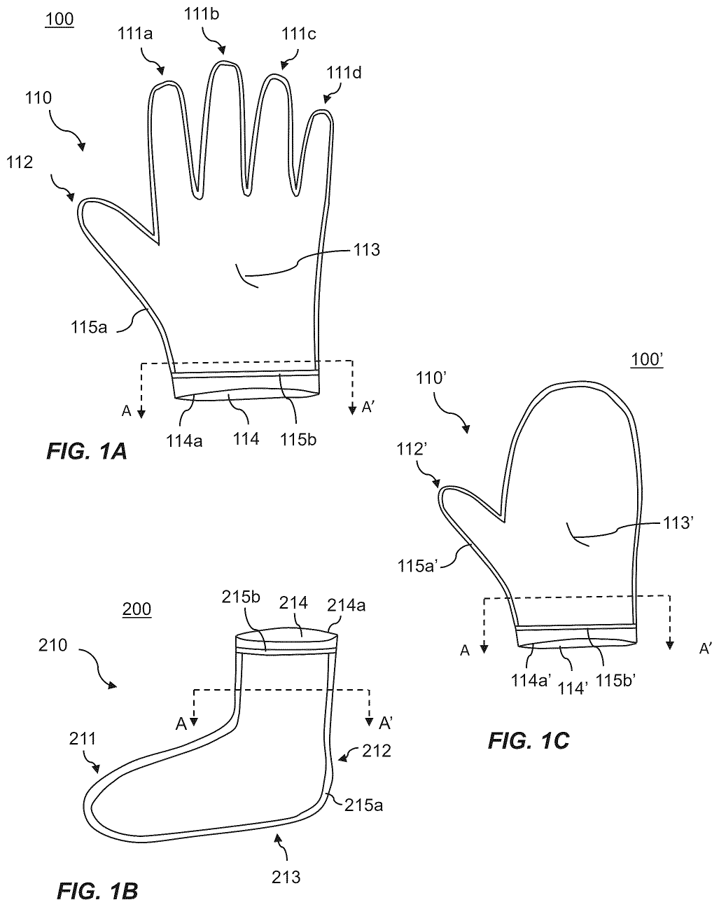

With reference to FIG. 1A, shown is a first embodiment of self-heating therapy system 100 of the disclosed invention, which includes a glove shape encaser structure 110. With reference to FIG. 1B, shown is a self-heating therapy system 200 of the disclosed invention, which includes a boot shape encaser structure 210. With reference to FIG. 1C, shown is a self-heating therapy system 100′ of the disclosed invention, which includes a mitt shape encaser structure 110′. The glove shape self-heating therapy system 100 includes finger portions 111a-111d, thumb portion 112 and palm/back region 113. The glove shape self-heating therapy system 100 is constructed to receive a hand in the internal space 114. The boot shape self-heating therapy system 200 includes toe region 211, ankle region 212 and sole and heel region 213. The boot shape self-heating therapy system 200 is constructed to receive a foot in the internal space 214. The mitt shape self-heating therapy system 100′ includes thumb portion 112′ and finger and palm/back region 113′. The mitt shape self-heating therapy system 100′ is constructed to receive a hand in the internal space 114′.

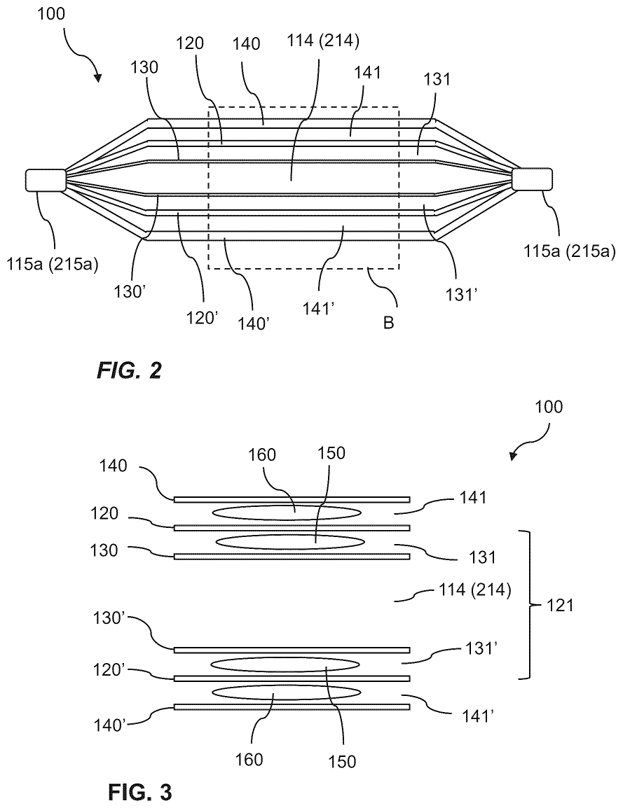

With reference to FIG. 2, shown is a cross-sectional view cut along A-A′ of the self-heating therapy systems 100, 200, and 100′ shown in FIGS. 1A-1C. With reference to FIG. 3, shown is layers of the section B of FIG. 2. The glove shape self-heating therapy system 100, the boot shape self-heating system 200, and the mitt shape self-heating therapy system 100′ have the same layer structure as shown in FIGS. 2-3, and therefore, the layer structure will be described referring to the glove shape self-heating therapy system 100.

In the first embodiment, the self-heating therapy system 100 includes warmer layers 140, 140′, encasers 120, 120′ disposed between the warmer layers 140 and 140′, and encaser liners 130, 130′ disposed between the encasers 120 and 120′. An interior space 121 is formed between the encasers 120, 120′, and the encaser lines 130, 130′ are disclosed inside the interior space 121. An internal space 114 to receive a hand is formed between the encaser liners 130 and 130′. Fillable space 131 is formed between the upper encaser 120 and upper encaser liner 130, and another fillable space 131′ is formed between the lower encaser 120′ and lower encaser liner 130′. The fillable spaces 131, 131′ are constructed to be able to contain materials. Warmer fillable space 141 is formed between the upper encaser 120 and the upper warmer layer 140, and another warmer fillable space 141′ is formed between the lower encaser 120′ and the lower warmer layer 140′. The warmer fillable spaces 141, 141′ are constructed to contain materials.

The self-heating therapy system 100 includes therapeutic composition 150 disposed in the fillable spaces 131, 131′ and warmer material 160 disposed in the warmer fillable space 141, 141′. The therapeutic composition 150 is for skin treatment and may include a wax-based mixture, butter-based mixture or solid-oils based mixture such as coconut oil. The therapeutic composition 150 may include mixtures of one or more of paraffin, coconut oil, vitamin E, and some additives. The warmer material 160 produces heat when exposed to air and may include a mixture of one or more of iron powder, water, salt, activated charcoal and wood fiber. The heat produced by the warmer material 160 is transferred to the therapeutic composition 150 through the encasers 120, 120′, so that the therapeutic composition 150 may be softened or melt by the heat produced by the warmer material 160.

In the descriptions, an inner surface of a layer, such as the warmer layer 140, encaser 120, and the encaser liner 130, is defined as a surface facing the internal space 114, and an outer surface of the layer is defined as a surface on the oppose side of the inner surface. Therefore, an inner surface of the encaser 120 and an outer surface of the encaser liner 130 define the fillable space 131, and an inner surface of the encaser 120′ and an outer surface of the encaser liner 130′ define the fillable space 131′. In the same way, an inner surface of the warmer layer 140 and an outer surface of the encaser 120 define the warmer fillable space 141, and an inner surface of the warmer layer 140′ and an outer surface of the encaser 120′ define the warmer fillable space 141′.

The therapeutic composition 150 may be a layer of a solidified or gel like material that may be softened or melt by heat, and may be placed in the fillable spaces 131, 131′ without any medium that may be saturated with the therapeutic composition. However, based on the materials of the therapeutic composition 150, a medium saturated with the therapeutic composition 150 may be placed in the fillable space 131, 131′.

The encaser liners 130, 130′ are made of a material that is permeable to one or more ingredients of the therapeutic composition 150. Therefore, the therapeutic composition 150, when softened or melt by heat, is transported to the internal space 114 through the encaser liners 130, 130′, and is applied to the skin of a hand that is received in the internal space 114. FIG. 3 exemplarily shows the therapeutic composition 150 placed in the fillable space 131, 131′ before migrating into the internal space 114. The encaser liner 130, 130′ may be made of material such as paper, textile, non-woven fabrics, plastic fabrics, non-woven polypropylene fabrics, and any combination thereof. In one embodiment, the encaser liner 130, 130′ is made of paper sheet. In another embodiment, the encaser liner 130, 130′ is made of non-woven polypropylene fabric configured to allow one or more ingredients of the therapeutic composition 150 to migrate from the fillable space 131, 131′ to the internal space 114 and to the user's skin during use. Additionally, the encaser liner 130, 130′ may provide a medium support or a holding agent for any form of the therapeutic composition 150 including, but not limited to, mud-based, clay-based, wax-based, liquid-based and gel-based compositions, such that the therapeutic composition 150 has reduced mobility within fillable space 131, 131′.

The encaser 120, 120′ may be made of any suitable encaser materials to contain the therapeutic composition 150 such as carbon-fiber, plastic, transparent/translucent polymer such as polyethylene, plastic film, metal foil, depending on design requirements and heating sources. The encaser 120, 120′ may be made of clastic material that allows for expanding or stretching. The material used to make the encaser 120, 120′ is heat-durable and does not release toxic chemicals upon heating. The encaser 120, 120′ is impermeable to the ingredients of the therapeutic composition 150. Therefore, the therapeutic composition 150 is not allowed to be transported to the warmer fillable space 141, 141′ through the encaser 120, 120′.

The warmer layers 140, 140′ are made of a material that is permeable to air. The warmer layers 140, 140′ may be made of materials such as porous non-woven cotton cloth. In an embodiment, the materials for the encaser liner 130, 130′ may be used for the warmer layers 140, 140′. For example, the warmer layers 140, 140′ may be made of non-woven polypropylene fabric. When the non-woven polypropylene fabric is used for the warmer layers 140, 140′, the structure shown in FIGS. 2-3, in which the warmer layers 140, 140′, the encasers 120, 120′, the encaser liner 130, 130′ are sealed together by the peripheral hermetic seal 115a (215a, 115a′), is preferred.

Before use, the self-heating therapy system 100 may be contained in a specially designed packaging (not shown) that may be sealed and in substantially vacuum state to prevent air infusion. Once the specially designed packaging is open and the self-heating therapy system 100 is taken from the packaging, the warmer layer 140, 140′ are exposed to air. Because the warmer layers 140, 140′ are air permeable, the exposure to air activates ingredients of the warmer material 160, producing heat. The encasers 120, 120′ are made of a material that is impermeable to both the therapeutic composition 150 and the warmer material 160, preventing any transportation of the therapeutic composition 150 and warmer material 160 through the encasers 120, 120′.

In the embodiment shown in FIG. 2, edges of the encasers 120, 120′, encaser liners 130, 130′, and warmer layers 140, 140′ are sealed or joined together by a peripheral hermetic seal 115a, 115a′, 215a, which is placed along a periphery of the glove shape structure 110, mitt shape structure 110′, and boot shape structure 210, respectively. Additionally, opening edges of the encasers 120, 120′, encaser liners 130, 130′, and warmer layers 140, 140′, which form the periphery of the opening to the internal space 114, may be sealed or joined together by an additional seal 115b, 115b′, 215b that is placed at a periphery of the opening 114a, 114a′, 214a or cuff to the internal space 114, 114′, 214, respectively.

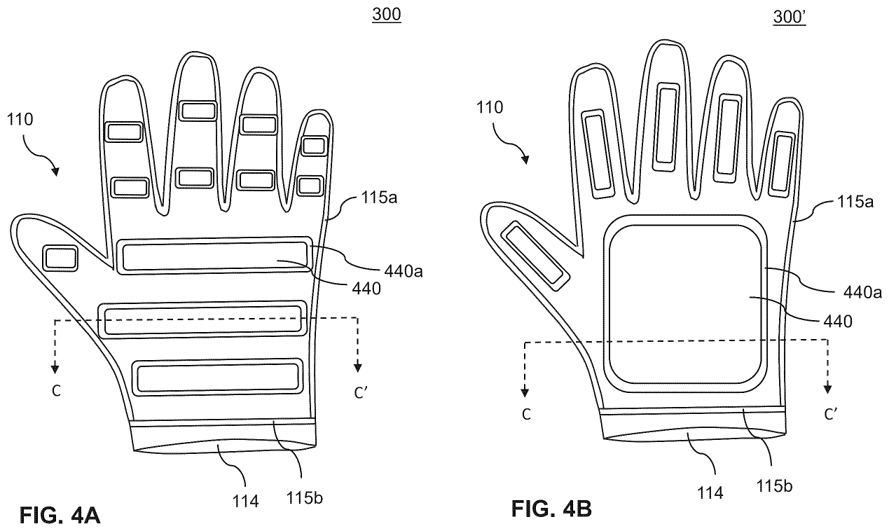

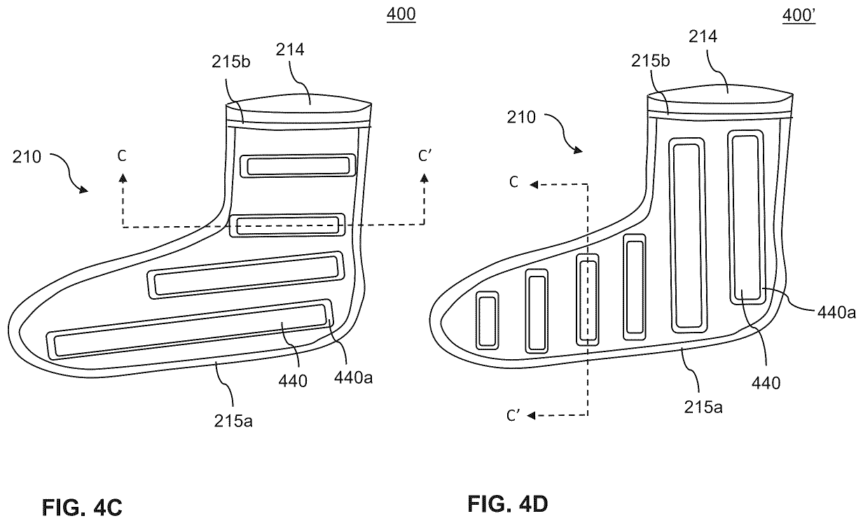

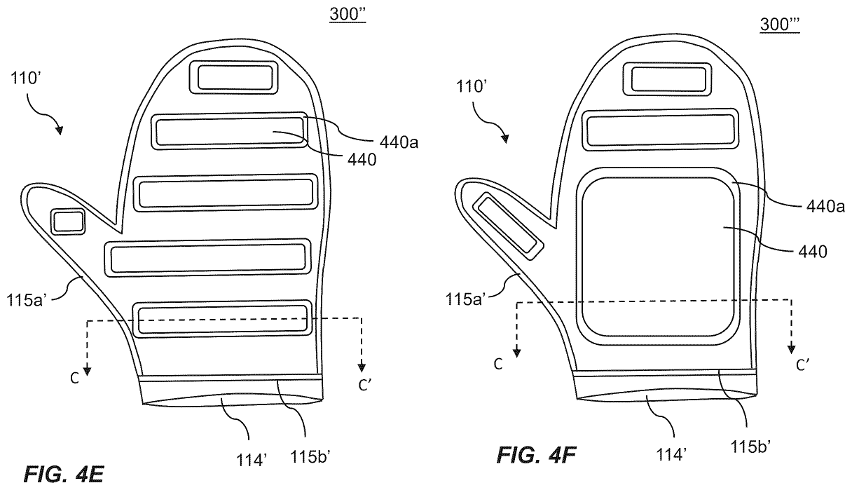

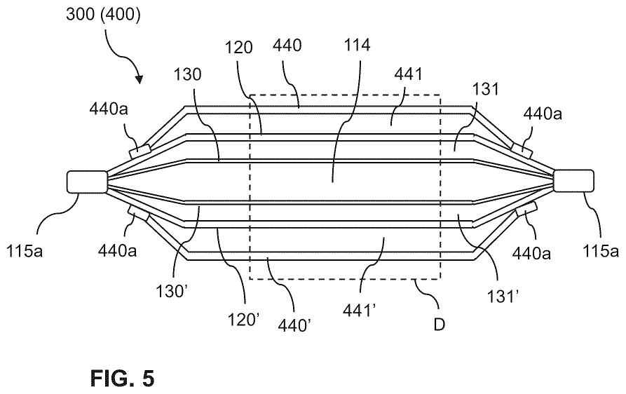

With reference to FIGS. 4A-4B, shown are embodiments of self-heating therapy systems 300, 300′ of the disclosed invention including a glove shape encaser structure 110. With reference to FIGS. 4C-4D, shown are embodiments of self-heating therapy systems 400, 400′ of the disclosed invention including a boot shape encaser structure 210. With reference to FIGS. 4E-4F, shown is a self-heating therapy system 300″, 300′″ of the disclosed invention, which includes a mitt shape encaser structure 110′. With reference to FIG. 5, shown is a cross-sectional view cut along C-C′ of the self-heating therapy systems 300, 300′, 400, 400′, 300″, 300″′ shown in FIGS. 4A-4F. The difference between the embodiments shown in FIGS. 4A-4F and the embodiments shown in FIGS. 1A-1C is the arrangements of the warmer layers.

As shown in FIGS. 4A-4F, the warmer layers 440 are arranged in various patterns. For example, FIGS. 4A, 4C, and 4E show horizontal strip patches of warmer layers 440. FIG. 4B exemplarily shows vertical strip patches of warmer layers 440 attached on finger portions and a rectangular shape patch of a warmer layer 440 attached on a palm/back area. FIG. 4D exemplarily shows vertical strip patches of warmer layers 440. FIG. 4F exemplarily shows strip patches of warmer layers 440 attached on the thumb and finger portions and a rectangular shape patch of a warmer layer 440 attached on a palm/back area. These warmer layers 440 are attached on an outer surface of the encaser 120 by adhesive layers 440a. FIGS. 4A-4F show some examples of patches of the warmer layers 440 attached on the encaser 120. The patches of the warmer layer 440 may have strip shapes, rectangular shapes, circular shapes, and/or oval shapes. The shapes and arrangements of the patches of the warmer layers 440, however, are not limited to those shown in FIGS. 4A-4F.

As shown in FIGS. 4A-4F and 5, the encasers 120, 120′ and encaser liners 130, 130′ are sealed by the peripheral hermetic seal 115a (215a, 115a′) and by the additional seal 115b (215b, 115b′) that is placed at a periphery of the opening to the internal space 114 (214). However, the warmer layers 440, 440′ are not sealed together with the encaser 120, 120′ and encaser liners 130, 130′. The warmer layers 440, 440′ are attached on the outer surfaces of the encasers 120, 120′ by using, for example, adhesives 440a. The layer structure of the section D is the same as the layer structure shown in FIG. 3. The patches of the warmer layers 440, 440′ may be separated from each other, and each patch defines the warmer fillable space 441 that contains warmer material 160. The fillable space 131 is filled with therapeutic composition 150, and the warmer fillable space 441 is filled with warmer materials 160.

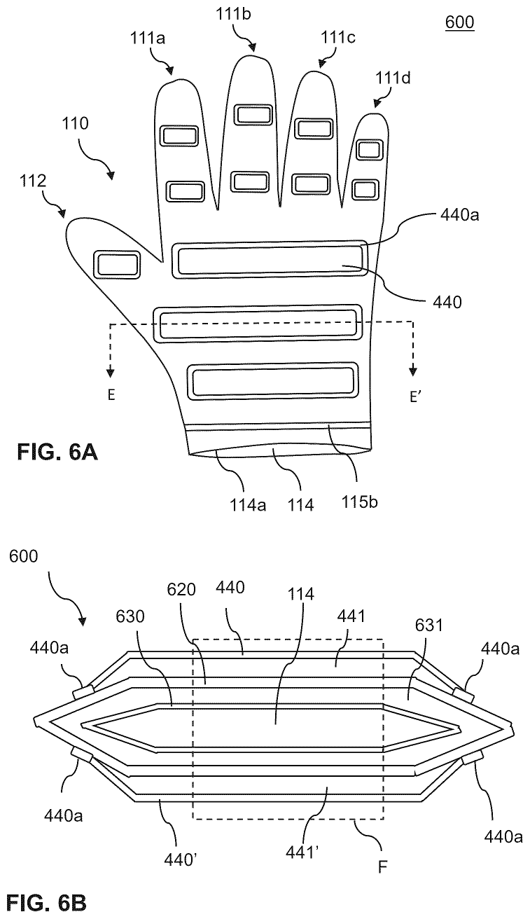

With reference to FIG. 6A, shown is an embodiment of the self-heating therapy system 600 of the disclosed invention including a glove shape encaser structure 110. With reference to FIG. 6B, shown is a cross-sectional view cut along E-E′ of the self-heating therapy system 600 shown in FIG. 6A. In this embodiment, the encaser 620 is formed as a single piece without the peripheral seal 115a along a periphery of the glove shape. The encaser liners 630 is also formed as a single piece, and is inserted in the interior space 121 of the encaser 120. However, the encaser 620 and the encaser liner 630 may be sealed together by the additional seal 115b that is placed at a periphery of the opening 114a to the internal space 114. The fillable space 631 is formed between the encaser 620 and the encaser liner 630, and is filled with therapeutic composition 150. The warmer layers 440, 440′ may be arranged to be attached on outer surfaces of the encaser 620 at upper side and lower side of the glove 110, respectively, by using, for example, adhesives 440a. The warmer fillable spaces 441, 441′ is filled with warmer materials 160. The layer structure of the section F is the same as the layer structure shown in FIG. 3. FIGS. 6A-6B exemplarily show glove shape self-heating therapy system 600, but the disclosed invention also provides a boot shape and mitt shape self-heating therapy systems that have the layer structure shown in FIG. 6B.

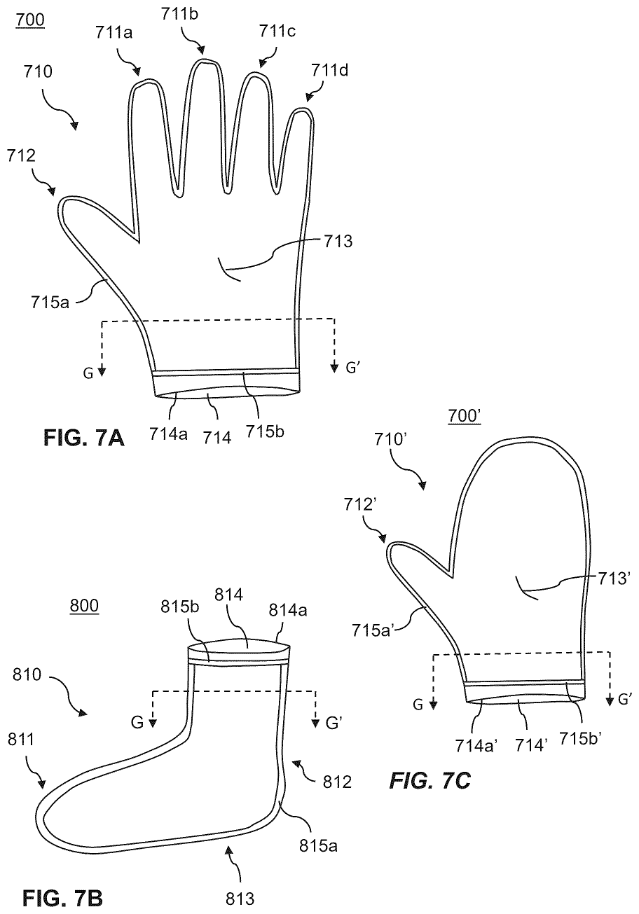

With reference to FIG. 7A, shown is second embodiment of self-heating therapy system 700 of the disclosed invention, which includes a glove shape encaser structure 710. With reference to FIG. 7B, shown is an embodiment of self-heating therapy system 800 of the disclosed invention, which includes a boot shape encaser structure 810. With reference to FIG. 7C, shown is an embodiment of self-heating therapy system 700′ of the disclosed invention, which includes a mitt shape encaser structure 710′. The glove shape self-heating therapy system 700 includes finger portions 711a-711d, thumb portion 712 and palm/back region 713. The glove shape self-heating therapy system 700 is constructed to receive a hand in the internal space 714. The boot shape self-heating therapy system 800 includes toe region 811, sole and ankle region 812 and heel region 813. The boot shape self-heating therapy system 800 is constructed to receive a foot in the internal space 814. The mitt shape self-heating therapy system 700′ includes thumb portion 712′ and palm/back region 713′. The mitt shape self-heating therapy system 700′ is constructed to receive a hand in the internal space 714′. The difference between the first embodiments shown in FIGS. 1A-6B and the second embodiments 700, 700′, 800 is the layer structure and ingredients inside the self-heating therapy system.

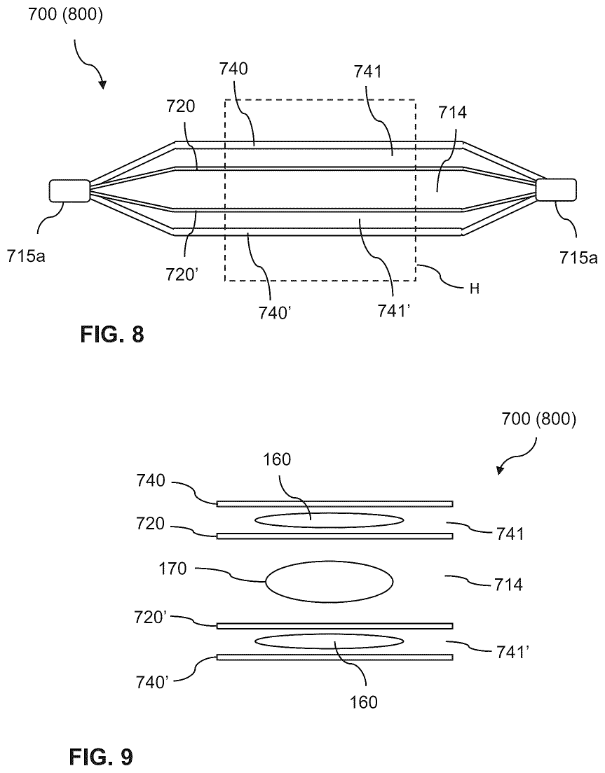

With reference to FIG. 8, shown is a cross-sectional view cut along G-G′ of the self-heating therapy systems 700, 800, and 700′ shown in FIGS. 7A-7C. With reference to FIG. 9, shown is a layer structure of the section H of FIG. 8. The glove shape self-heating therapy system 700, the boot shape self-heating system 800, and the mitt shape self-heating therapy system 700′ have the same layer structure as shown in FIGS. 8-9. Therefore, the layer structure will be described referring to the glove shape self-heating therapy system 700.

In the second embodiment, the self-heating therapy system 700 includes encasers 720, 720′ and warmer layers 740, 740′ on the upper and lower sides of the glove shape structure 710, respectively. An internal space 714 to receive a hand is formed between the encaser 720 and 720′. Warmer fillable space 741 is formed between the upper encaser 720 and the upper warmer layer 740, and another warmer fillable space 741′ is formed between the lower encaser 720′ and the lower warmer layer 740′. The fillable spaces 741, 741′ are constructed to be able to contain materials.

The self-heating therapy system 700 includes skincare composition 170 disposed in the internal space 714 and warmer material 160 disposed in the warmer fillable spaces 741 and 741′. The skincare composition 170 may include a mixture of one or more of antioxidant, lotion, and therapy ingredients. The skincare composition 170 may include butter-based mixture or solid-oils based mixture such as coconut oil. As described above, the warmer material 160 produces heat when exposed to air. The heat produced by the warmer material 160 is transferred to the skincare composition 170 through the encaser 720, 720′, so that the skincare composition 170 is softened by the heat and may be easily spread on a hand that is received in the internal space 714.

As described above, the warmer layers 740, 740′ are made of a material that is permeable to air, and the encasers 720, 720′ are made of a material that is impermeable to both the skincare composition 170 and the warmer material 160, preventing any transportation of the skincare composition 170 and warmer material 160 through the encasers 720, 720′. The encaser 720 may be made of any suitable encaser materials to contain the skincare composition 170, such as carbon-fiber, plastic, transparent/translucent polymer such as polyethylene, plastic film, metal foil, depending on design requirements and heating sources. The encaser 720 may be made of clastic material that allows for expanding or stretching. The material used to make the encaser 720 is heat-durable and does not release toxic chemicals upon heating. The warmer layers 740, 740′ are made of a material that is permeable to air. The warmer layers 740, 740′ may be made of materials such as porous non-woven cotton cloth. The warmer layers 740 may be made of the same materials used for the encaser liner 130, 130′ in the first embodiment shown in FIGS. 1A-6B.

In an embodiment shown in FIG. 8, the encasers 720, 720′ and warmer layers 740, 740′ may be sealed or joined together by a peripheral hermetic seal 715a (815a, 715a′), which is placed along a periphery of the glove shape structure 710 or boot shape structure 810. Additionally, the encasers 720, 720′ and warmer layers 740, 740′ may be sealed or joined together by an additional seal 715b (815b, 715b′) that is placed at a periphery of the opening 714a (814, 714a′) to the internal space 714 (814, 714′).

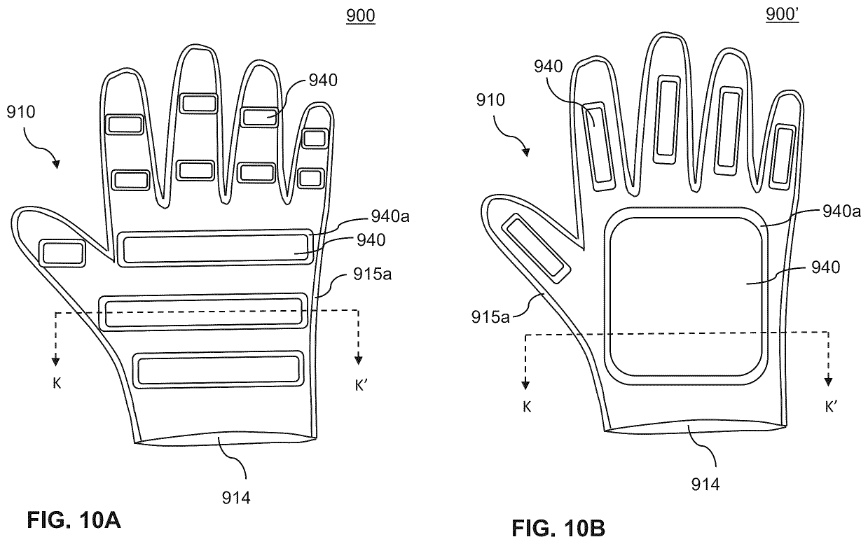

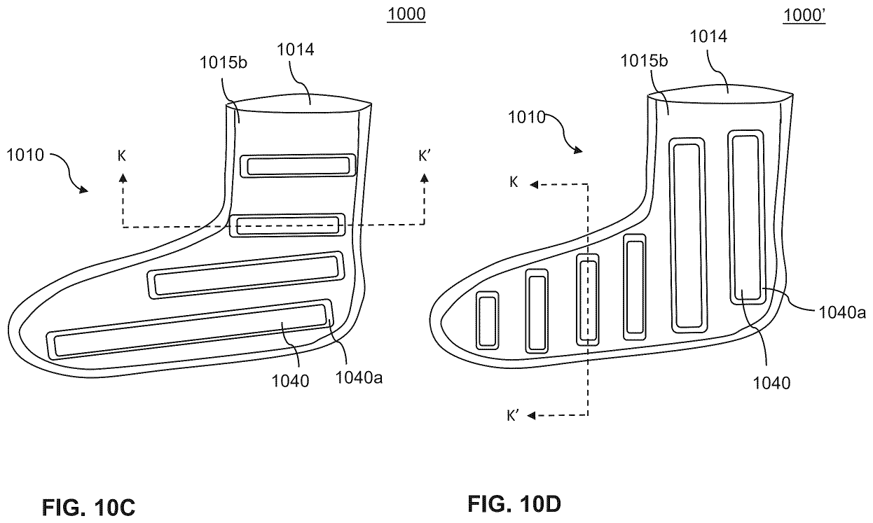

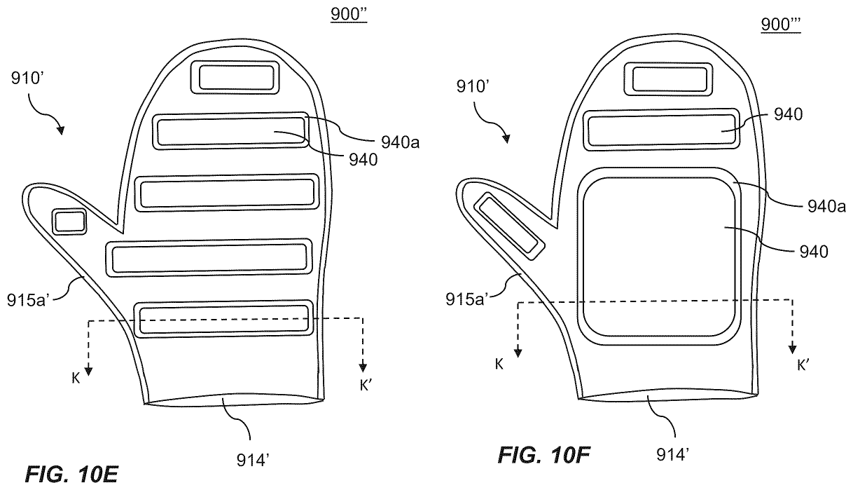

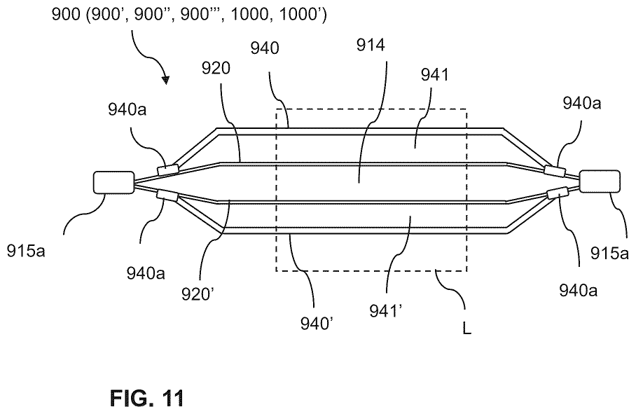

With reference to FIGS. 10A-10B, shown are embodiments of the self-heating therapy systems 900, 900′ of the disclosed invention including a glove shape encaser structure 910. With reference to FIGS. 10C-10D, shown are embodiments of the self-heating therapy systems 1000, 1000′ of the disclosed invention including a boot shape encaser structure 1010. With reference to FIGS. 10E-10F, shown are embodiments of the self-heating therapy systems 900″, 900″′ of the disclosed invention including a mitt shape encaser structure 910′. With reference to FIG. 11, shown is a cross-sectional view cut along K-K′ of the self-heating therapy systems 900, 900′, 1000, 1000′, 900″, 900′″ shown in FIGS. 10A-10F. The difference between the embodiments shown in FIGS. 10A-10F and the embodiments shown in FIGS. 7A-7C is the arrangements of the warmer layers.

As shown in FIGS. 10A-10F, the warmer layers 940, 1040 are arranged in various patterns. For example, FIGS. 10A, 10C, and 10E show horizontal strip patches of warmer layers 940, 1040. FIG. 10B exemplarily shows strip patches of warmer layers attached on finger portions and a rectangular shape patch of a warmer layer attached on a palm/back area. FIG. 10D exemplarily shows vertical strip patches of warmer layers. FIG. 10F exemplarily shows strip patches of warmer layers attached on the thumb and finger portions and a rectangular shape patch of a warmer layer attached on a palm/back area. These warmer layers 940, 1040 are attached on outer surface of the encaser 920 by adhesive layers 940a. FIGS. 10A-10F show examples of the warmer layers 940 attached on the encaser 920. The shapes and arrangements of the warmer layers 940, however, are not limited to those shown in FIGS. 10A-10F.

As shown in FIGS. 10A-10F and 11, the encasers 920, 920′ are sealed by the peripheral hermetic seal 915a (1015a, 915a′). However, the warmer layers 940, 940′ are not sealed together with the encaser 920, 920′. The warmer layers 940, 940′ are attached on the outer surfaces of the encasers 920, 920′ by using, for example, adhesives 940a. The layer structure of the section L is the same as the layer structure shown in FIG. 9. The internal space 914 (1014, 914′) is filled with skincare composition 170, and the warmer fillable spaces 941, 941′ are filled with warmer materials 160.

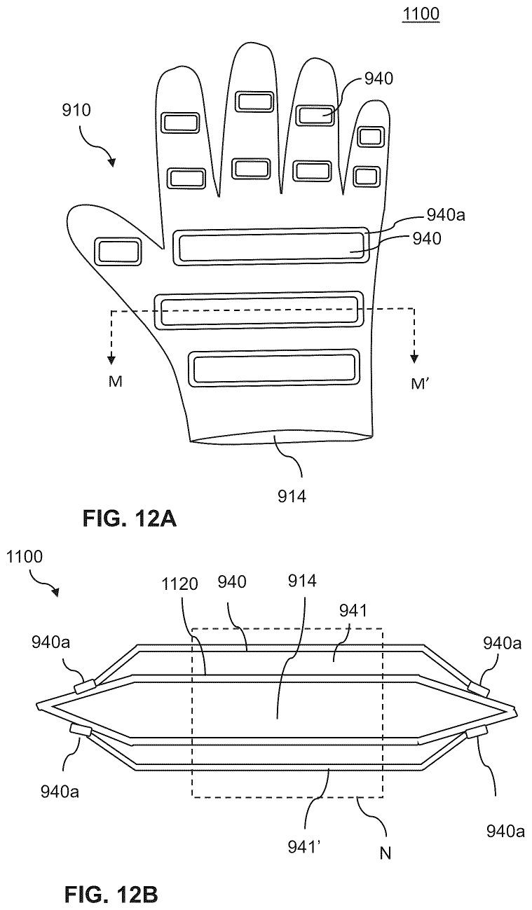

With reference to FIG. 12A, shown is another embodiment of the self-heating therapy systems 1100 of the disclosed invention including a glove shape encaser structure 910. With reference to FIG. 12B, shown is a cross-sectional view cut along M-M′ of the self-heating therapy system 1100 shown in FIG. 12A. In this embodiment, the encaser 1120 is formed as a single piece, without the peripheral seal 1015a along a periphery of the glove shape. The internal space 914 is filled with skincare composition 170, and the warmer fillable spaces 941, 941′ are filled with warmer materials 160. The layer structure of the section N is the same as the layer structure shown in FIG. 9. FIGS. 12A-12B exemplarily show glove shape self-heating therapy system 1100, but the disclosed invention also provides a boot shape and mitt shape self-heating therapy systems that have the layer structure shown in FIG. 12B.

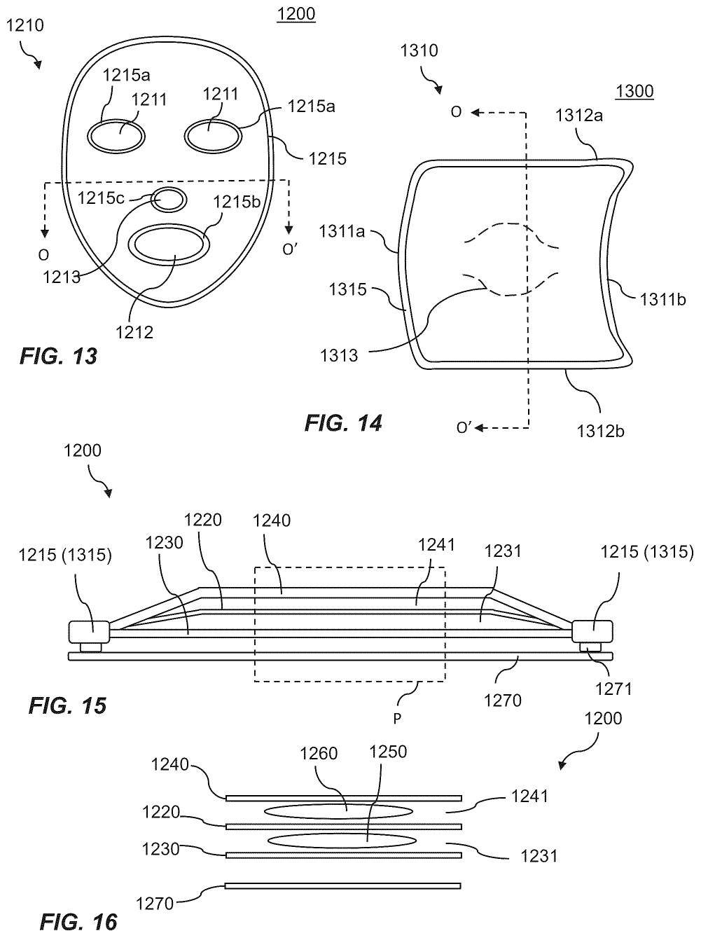

With reference to FIG. 13, shown is a third embodiment of self-heating therapy system 1200 of the disclosed invention including a face mask shape encaser structure 1210. The self-heating therapy systems 1200 is constructed to substantially cover the face of a wearer with open (cut-out) eye portions 1211, open mouth portion 1212, and open nostril portion 1213 to expose the eyes, nostril, and mouth regions of the wearer, respectively. The self-heating therapy system 1200 may have straps (not shown) connected to edges of the face mask shape encaser structure 1210 to tie the face mask shape encaser structure 1210 around head portion of the wearer. Alternatively, the face mask shape encaser structure 1210 may have adhesive layers (not shown) to attach the face mask shape encaser structure 1210 on the face of the wearer.

With reference to FIG. 14, shown is an embodiment of self-heating therapy system 1300 of the disclosed invention including a knee or elbow shape encaser structure 1310. The self-heating therapy systems 1300 is constructed to substantially cover or enclose the knee portion or elbow portion of a wearer. The knee or elbow shape encaser structure 1310 may be a cuff or band shape structure that may include a swollen central portion 1313 that is constructed to cover the knee or elbow of a wearer. Therefore, the knee or elbow shape encaser structure 1310 has a shape suitable to cover or wrap elbow or knee of a user. The vertical edges 1311a, 1311b may be curved edges to wrap the knee or elbow portion of the wearer. The knee or elbow shape encaser structure 1310, may enclose the knee or elbow regions of the wearer by moving the horizontal edges 1312a, 1312b around the knee or elbow regions of the wearer, while the central portion 1313 is placed at the knee or elbow of the wearer. The self-heating therapy system 1300 may have straps (not shown) connected to the horizontal edges 1312a, 1312b to tie the knee or elbow shape encaser structure 1310 around the knee or elbow region of the wearer. Alternatively, the knee or elbow shape encaser structure 1310 may have adhesive layers (not shown) to attach the knee or elbow shape encaser structure 1310 on the knee or elbow region of the wearer.

With reference to FIG. 15, shown is a cross-sectional view cut along O-O′ of the self-heating therapy systems 1200, 1300 shown in FIGS. 13-14. With reference to FIG. 16, shown is layers of the section P of FIG. 15. The face mask shape self-heating therapy system 1200 and the knee or elbow shape self-heating system 1300 have the same layer structure as shown in FIGS. 15-16, and therefore, the layer structure will be described referring to the face mask shape self-heating therapy system 1200. The difference between the self-heating therapy systems 1200, 1300 and the other embodiments shown in FIGS. 1A-12 is that the self-heating therapy systems 1200, 1300 do not have internal space to receive a body part. The self-heating therapy system 1200, 1300 are configured to be placed on the body part of the wearer.

The self-heating therapy system 1200, 1300 include encaser liner 1230, encaser 1220 disposed above the encaser liner 1230, and warmer layer 1240 disposed above the encaser 1220. Fillable space 1231 is formed between the encaser 1220 and the encaser liners 1230. The fillable spaces 1231 is constructed to be able to contain materials. Warmer fillable space 1241 is formed between the encaser 1220 and the warmer layer 1240. The warmer fillable space 1241 is constructed to contain materials. The encaser liner 1230, the encaser 1220, and warmer layer 1240, may be made of the same materials as the encaser liner 130, the encaser 120, and warmer layer 140, respectively, as described above.

In the embodiments shown in FIGS. 13-16, the encaser liner 1230, the encaser 1220, and warmer layer 1240 are sealed together by the peripheral hermetic seal 1215, 1315. In the face mask shape self-heating therapy system 1200, the seals 1215a, 1215b, 1215c may be formed on the peripheries of the eye portions 1211, mouth portion 1212, nostril portion 1213, respectively.

The self-heating therapy system 1200, 1300 further include peel-off layer 1270 disposed below the encaser liner 1230 to prevent any leakage of the composition 1250 before use. The peel-off layer 1270 may be attached to the seal 1215 by an adhesive layer 1271 or by other means known in the art. When the self-heating therapy system 1200, 1300 are to be used, the peel-off layer 1270 is removed, and the encaser liner 1230 directly contacts face, knee, or elbow region of a user.

The self-heating therapy system 1200 includes composition 1250 disposed in the fillable space 1231 and warmer material 1260 disposed in the warmer fillable space 1241. The composition 1250 may be the therapeutic composition 150 for skin treatment or the skincare composition 170 described above referring to FIGS. 1A-12. The warmer material 1260 may be the same warmer material 160 described above, and produces heat when exposed to air. Once the specially designed packaging is open and the self-heating therapy system 1200, 1300 is taken from the packaging, the warmer layer 1240 is exposed to air, and the warmer material 1260 begins to produce heat, as air enters the warmer fillable space 1241 through the warmer layer 1240. The heat produced by the warmer material 1260 is transferred to the composition 1250 through the encaser 1220, so that the composition 1250 may be softened or melt by the heat from the warmer material 1260. The encaser liner 1230 is made of a material that is permeable to one or more ingredients the composition 1250. Therefore, the composition 1250, when softened by heat, is transported through the encaser liner 1230, and is applied to the skin of the face, knee, and elbow region of the user.

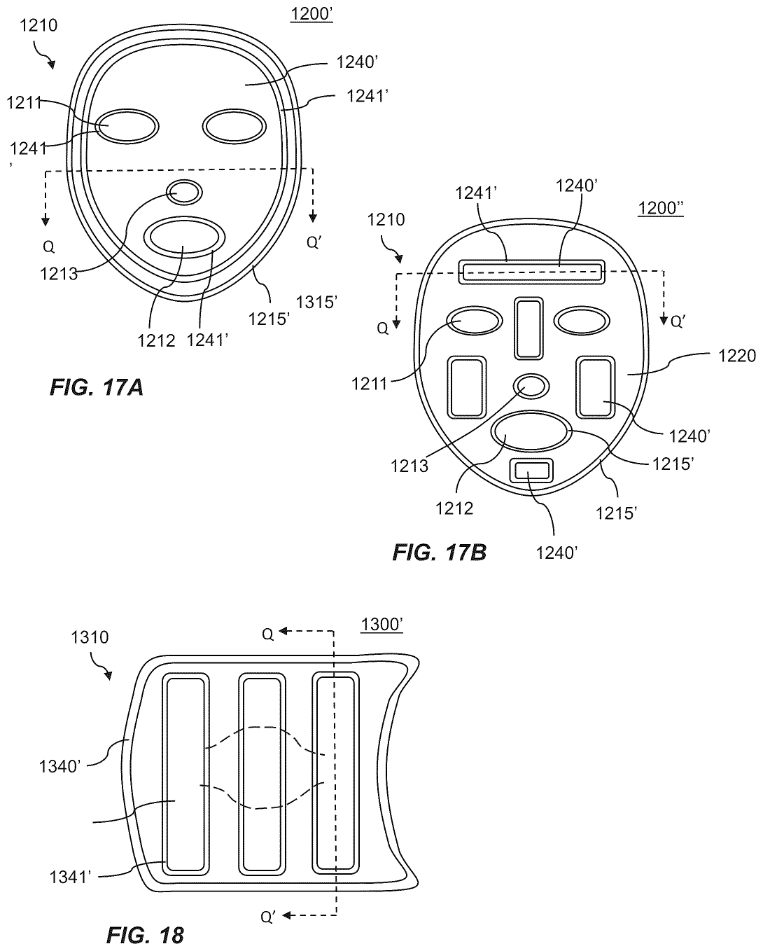

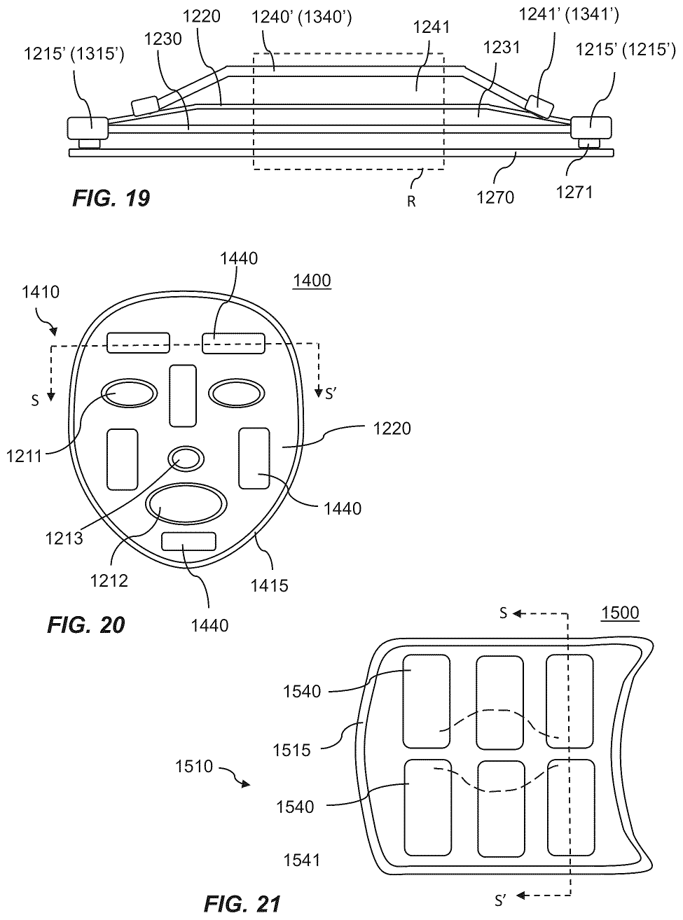

With reference to FIGS. 17A-17B, shown are another embodiment of the self-heating therapy system 1200′ and 1200″ of the disclosed invention including a face mask shape encaser structure 1210. With reference to FIG. 18, shown is another embodiment of the self-heating therapy system 1300′ of the disclosed invention including a knee or elbow shape encaser structure 1310. With reference to FIG. 19, shown is a cross-sectional view cut along Q-Q′ of the self-heating therapy systems 1200′, 1300′ shown in FIGS. 17A-18. In the self-heating therapy system 1200′, 1200″, 1300′, the warmer layer 1240′, 1340′ is attached on an outer surface of the encaser 1220 by an adhesive layer 1241′, 1341′, while the encaser 1220 and the encaser liner 1230 are sealed together by seal 1215′, 1315′. The layers of the section R are the same as the layers shown in FIG. 16. FIG. 17A shows a single patch of the warmer layer 1240′ substantially covering the face of a user. FIG. 17B shows a plurality of patches distributed over the face of the user.

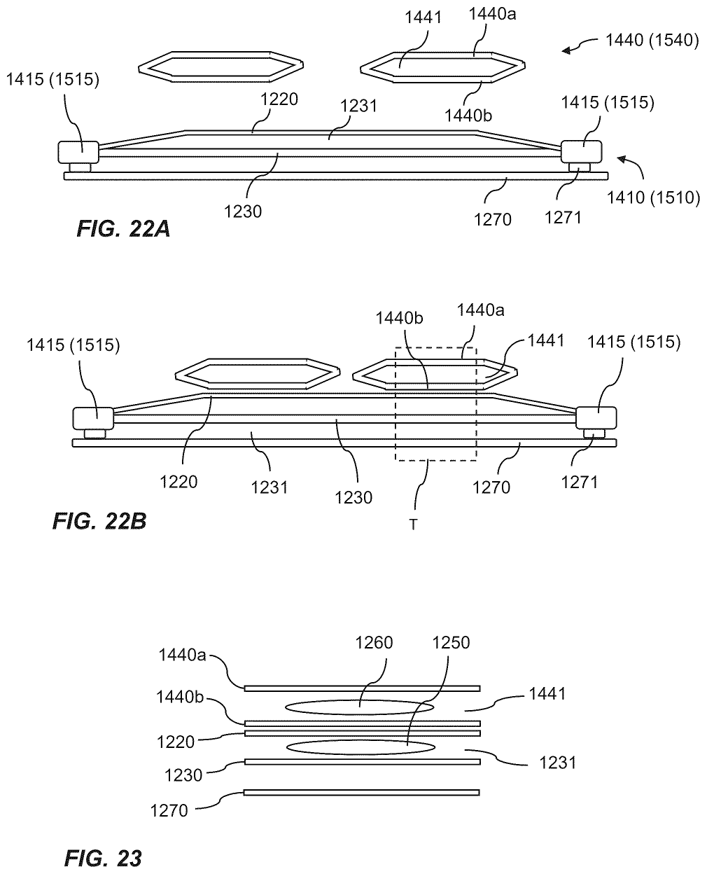

With reference to FIG. 20, shown is a fourth embodiment of self-heating therapy system 1400 of the disclosed invention including a face mask shape encaser structure 1410 and warmer pack 1440. With reference to FIG. 21, shown is an embodiment of self-heating therapy system 1500 of the disclosed invention including a knee or elbow shape encaser structure 1510 and warmer pack 1540. With reference to FIGS. 22A-22B, shown are cross-sectional views cut along S-S′ of the self-heating therapy systems 1400, 1500 shown in FIGS. 20-21. With reference to FIG. 23, shown is layers of the section T of FIG. 22B.

In the embodiment shown in FIGS. 20-23, the self-heating therapy systems 1400, 1500 include warmer packs 1440, 1540, respectively, instead of the warmer layers 1240′, 1340′ shown in FIGS. 17-19. The warmer packs 1440, 1540 contain the warmer material 1260 in the warmer fillable space 1441. In this embodiment, the encaser structures 1410, 1510 and the warmer packs 1440, 1540 may be provided in separate packages or pouches. The warmer packs 1440, 1540 are constructed to be attachable to the encaser structures 1410, 1510 to provide heat to the encaser structures when the warmer packs are activated. FIG. 22A shows the cross-sectional view in which the warmer pack 1440 (1540) is separately provided before the warmer pack 1440 (1540) is attached to the encaser structure 1410, 1510. FIG. 22B shows the cross-sectional view in which the warmer pack 1440 (1540) is attached to the encaser structure 1410 (1510).

The warmer packs 1440 (1540) includes upper layer 1440a and lower layer 1440b. The upper layer 1440a is made of a material that is permeable to air such as porous non-woven cotton cloth, as described above referring to FIGS. 2-3. The lower layer 1440b is constructed to be attached to the encaser 1220 of the encaser structure 1410 (1510). An adhesive layer (not shown) or any attachable member may be formed on a lower surface of the lower layer 1440b, which faces the encaser 1220, to securely attach the warmer pack 1440 (1540) to the encaser structure 1410 (1510) as shown in FIG. 23. The features of the encaser 1220, encaser liner 1230, fillable space 1231, composition 1250, and peel-off layer 1270 are the same as described above referring to FIGS. 13-19.

In practical applications, the face mask shape encaser structure 1410 and the knee or elbow shape encaser structure 1510 may be provided inside a pouch in a wet environment. Therefore, providing the encaser structures 1410, 1510 in a separate pouch from the warmer pack 1440, 1540 may have advantages to prevent contamination of the warmer material in the warmer pack and accidental activation of the warmer material before intended use. When the self-heating therapy system is used, a user may lay, for example, the face mask shape encaser structure 1410 on the face and then lay the warmer pack 1440 over the face mask shape encaser structure 1410.

FIG. 20 exemplarily shows multiple rectangular shape warmer packs 1440 attached on the encaser 1220. However, the shape of the warmer packs 1440 is not limited to the shapes shown in FIG. 20, but may have different shapes to properly heat the encaser structure 1410 when activated. For example, the warmer pack 1440 may include a certain shape of a warmer pack and/or multiple various shape warmer sub-packs, each of which includes warmer materials.

Since many modifications, variations, and changes in detail can be made to the described preferred embodiments of the invention, it is intended that all matters in the foregoing description and shown in the accompanying drawings be interpreted as illustrative and not in a limiting sense. Consequently, the scope of the invention should be determined by the appended claims and their legal equivalents.

What is claimed is:

1. A self-heating skin therapy system for skin conditioning or treatment of a body part, comprising:
a body part shaped encaser having an interior space;
an encaser liner disposed in the interior space of the encaser, wherein the encaser liner has an internal space configured to receive the body part, and wherein an inner surface of the encaser and an outer surface of the encaser liner are configured to define a first fillable space;
at least one warmer layer disposed on an outer surface of the encaser, wherein an inner surface of the warmer layer and the outer surface of the encaser are configured to define a second fillable space;
a therapeutic composition contained within the first fillable space, wherein the encaser liner is permeable to one or more ingredients of the therapeutic composition; and
a warmer material contained in the second fillable space, wherein the warmer layer is permeable to air and the warmer material produces heat when exposed to air.

2. The self-heating skin therapy system of claim 1 wherein the encaser is impermeable to both the therapeutic composition and the warmer material.

3. The self-heating skin therapy system of claim 1 wherein the at least one warmer layer is attached on the outer surface of the encaser.

4. The self-heating skin therapy system of claim 3 further comprising a peripheral seal by which edges of the encaser and the encaser liner are joined together.

5. The self-heating skin therapy system of claim 3 wherein the at least one warmer layer comprises a plurality of patches attached on the outer surface of the encaser, and each patch defines a warmer fillable space filled with the warmer material.

6. The self-heating skin therapy system of claim 5 wherein the patches have one or more shapes selected from the group consisting of a strip shape, rectangular shape, circular shape, and oval shape.

7. The self-heating skin therapy system of claim 1 further comprising a peripheral seal by which edges of the encaser, the encaser liner and the warmer layer are joined together.

8. The self-heating skin therapy system of claim 1 wherein the warmer material comprises a mixture of one or more selected from the group consisting of iron powder, water, salt, activated charcoal and wood fiber.

9. The self-heating skin therapy system of claim 1 wherein the encaser liner includes non-woven polypropylene fabric.

10. The self-heating skin therapy system of claim 1 wherein the warmer layer includes cotton cloth or non-woven polypropylene fabric.

11. The self-heating skin therapy system of claim 1 wherein the body part shaped encaser has a shape selected from the group consisting of a glove shape, boot shape, and a mitt shape.

12. A self-heating skin therapy system for skin conditioning or treatment of a body part, comprising:
a body part shaped encaser having an internal space configured to receive the body part;
at least one warmer layer disposed on an outer surface of the encaser, wherein an inner surface of the warmer layer and the outer surface of the encaser are configured to define a fillable space;
a skincare composition contained within the internal space; and
a warmer material contained in the fillable space, wherein the warmer layer is permeable to air and the warmer material produces heat when exposed to air.

13. The self-heating skin therapy system of claim 12 wherein the encaser is impermeable to both the skincare composition and the warmer material.

14. The self-heating skin therapy system of claim 12 wherein the at least one warmer layer is attached on the outer surface of the encaser.

15. The self-heating skin therapy system of claim 14 wherein the at least one warmer layer comprises a plurality of patches attached on the outer surface of the encaser, and each patch defines a warmer fillable space filled with the warmer material.

16. The self-heating skin therapy system of claim 12 further comprising a peripheral seal by which edges of the encaser and the warmer layer are joined together.

17. A self-heating skin therapy system for skin conditioning or treatment of a body part, comprising:
a body part shaped encaser;
an encaser liner disposed below the encaser, wherein a lower surface of the encaser and an upper surface of the encaser liner are configured to define a first fillable space, and wherein a lower surface of the encaser liner is configured to contact a skin of a user;
at least one warmer layer disposed above the encaser, wherein a lower surface of the warmer layer and an upper surface of the encaser are configured to define a second fillable space;
a composition contained within the first fillable space, wherein the encaser liner is permeable to one or more ingredients of the composition; and
a warmer material contained in the second fillable space, wherein the warmer layer is permeable to air and the warmer material produces heat when exposed to air.

18. The self-heating skin therapy system of claim 17 wherein the encaser is impermeable to both the therapeutic composition and the warmer material.

19. The self-heating skin therapy system of claim 17 wherein the at least one warmer layer is attached on the outer surface of the encaser.

20. The self-heating skin therapy system of claim 19 wherein the at least one warmer layer comprises a plurality of patches attached on the outer surface of the encaser, and each patch defines a warmer fillable space filled with the warmer material.

21. The self-heating skin therapy system of claim 19 further comprising a peripheral seal by which edges of the encaser and the encaser liner are joined together.

22. The self-heating skin therapy system of claim 17 wherein the body part shaped encaser has a shape suitable to cover face, elbow or knee of a user.
